(19) United States
(12) Patent Application Publication
(10) Pub. No.: US 2021/0275274 A1
(43) Pub. Date: Sep. 9, 2021

(54) Fixed Camera Apparatus, System, and Method for Facilitating Image-Guided Surgery

(71) Applicants: John B. Clayton, Superior, CO (US); Kevin T. Foley, Germantown, TN (US)

(72) Inventors: John B. Clayton, Superior, CO (US); Kevin T. Foley, Germantown, TN (US)

(21) Appl. No.: 17/132,268

(22) Filed: Dec. 23, 2020

Related U.S. Application Data
(60) Provisional application No. 62/985,673, filed on Mar. 5, 2020.

(51) Int. Cl. A61B 90/14; G03B 17/56; F16M 13/02; F16M 13/04; A61B 90/50

(52) U.S. Cl. CPC A61B 90/14; G03B 17/561; A61B 2090/502; F16M 13/04; A61B 90/50; F16M 13/022

(57) ABSTRACT

An apparatus, system, and method for providing a rigid connection between a localizing camera and a patient's head for image-guided surgery. A fixed camera head clamp includes a camera mount for rigidly securing a localizing camera to the head clamp. In one embodiment, a proximal end of the camera mount is selectively secured to a receptacle of a frame of the head clamp and a distal end of the camera mount includes a camera receptacle configured to receive a localizing camera. A fixed camera head clamp system includes the head clamp and a localizing camera. A camera mount system includes a camera mount secured to a patient's head and a localizing camera secured to a camera receptacle on the camera mount.

### CROSS REFERENCE TO RELATED APPLICATION

This application is a non-provisional, and claims the benefit, of U.S. Provisional Patent Application No. 62/985,673, filed on Mar. 5, 2020, which is incorporated herein by reference in its entirety.

### BACKGROUND

In the field of image-guided cranial surgery, a localizing camera in the operating room tracks the positions of surgical tools in three-dimensional space. This positional information is transmitted from the localizing camera to a computer. The computer's monitor displays multiplanar, three-dimensional radiographic images of the patient's head and brain that have been uploaded into the computer. The patient's cranial anatomy as positioned in the operating room is registered to the radiographic image data using a probe that is tracked by the localizing camera. In image-guided surgery, registration is the process that transforms the three-dimensional radiographic data set (image space) so that it correlates with the three-dimensional coordinates of the corresponding patient anatomy (surgical space). Following registration of the cranial anatomy, the navigation system can display the positions of the tracked surgical tools relative to the displayed radiographic anatomy. In order for this process to be accurate, the three-dimensional spatial relationship between the localizing camera and the patient's head must be known and maintained. If the localizing camera moves or if the patient's head moves during surgery, the navigation system's accuracy deteriorates. In order to compensate for this issue, a tracked tool known as a dynamic reference frame is fixed in relationship to the patient's head. If the patient's head and the localizing camera move relative to one another, their three-dimensional relationship is recalculated by the navigation system computer and the registration solution is updated. Typically, the dynamic reference frame is fixed to the clamp that holds the patient's head in a stationary position for surgery.

While the above measures can serve to maintain the accuracy of image-guided cranial surgery, they have limitations. In order to track the dynamic reference frame, the localizing camera measures the spatial coordinates of markers on the reference frame in the x-axis, y-axis, and z-axis. The accuracy of these measurements changes as the viewing angle changes between the camera and the markers. It is a common practice to move the camera to maintain its line of sight for localizing the dynamic reference frame to accommodate such things as microscopes, various instruments, and additional operating room personnel entering the surgical field. As well, the patient's head is often repositioned during brain surgery, such as occurs when elevating the head of the operating room table, rotating the table, and raising or lowering the table. In these instances, navigational error is inadvertently induced when the camera moves relative to the markers on the dynamic reference frame.

Conventional head clamps include loose fittings that allow the patient's head to move in the head clamp. When the localizing camera is tracking the dynamic reference frame, the navigation system assumes that there is no movement of the head relative to the reference frame. However, in actual use there may be as much as 4 mm of head movement relative to the head clamp (and thus to the dynamic reference frame fixed to the head clamp). The error introduced by this relative movement is not detected by the navigation system and does not provide the optimal accuracy needed in cranial surgery.

Additionally, the head clamp is nonsterile and requires a sterile drape to maintain a sterile field during cranial surgery. In typical image-guided cranial surgery, the registration process is carried out prior to the application of the sterile drape because the drape obscures portions of the patient's cranial anatomy that need to be visible and accessible during registration. A nonsterile dynamic reference frame is used during this process. Following registration, the nonsterile dynamic reference frame is removed, the patient's head is prepared and draped in sterile fashion, and a sterile dynamic reference frame is attached to the head clamp through the drapes. Errors are inadvertently introduced when the nonsterile dynamic reference frame is removed and replaced with a sterile dynamic reference frame.

There remains a need for instruments and techniques that minimize or eliminate relative motion between the localizing camera and the dynamic reference frame during image-guided cranial surgery. There further remains a need to minimize line of sight issues during these surgeries.

### SUMMARY

Devices, systems, and methods are provided that are designed to minimize relative motion between a localizing camera and a patient's head during cranial surgery. An improved head clamp is provided that minimizes the relative motion between the clamp and the patient's head. A small localizing camera is provided that is rigidly mountable to the improved head clamp such that relative motion between the clamp and the localizing camera is eliminated, the need for a dynamic reference frame is eliminated, and line of sight issues are minimized during image-guided surgery. A camera mount system is provided that is designed to be secured directly to a patient's head. Methods for improving the accuracy of image-guided surgery by utilizing the improved head clamp and the rigidly mountable localizing camera, and by using the camera mount system, are provided.

### BRIEF DESCRIPTION OF THE DRAWINGS

FIG. 1 is a perspective view of a fixed camera head clamp for providing a rigid connection between a localizing camera and a patient's head for image-guided surgery.

FIG. 2 is an exploded view of the fixed camera head clamp.

FIG. 3 is a detail perspective view of a receptacle of the fixed camera head clamp.

FIG. 4 is a detail perspective view of a proximal end of a camera mount of the fixed camera head clamp.

FIG. 5 is a section view of the receptacle and the proximal end of the camera mount taken from line 5-5 in FIG. 1.

FIG. 6 is a detail perspective view of a distal end of the camera mount.

FIG. 7 is a perspective view of a fixed camera head clamp system including a camera attached to the camera mount.

FIG. 8 is a perspective view of the fixed camera head clamp system with a drape over the camera and camera mount.

FIG. 9 is a chart comparing the accuracy loss resulting from various activities using a conventional head clamp and using the fixed camera head clamp.

FIG. 10 is a perspective view of a camera mount system for providing a rigid connection between a localizing camera and a patient's head for image-guided surgery.

### DETAILED DESCRIPTION OF THE PREFERRED EMBODIMENTS

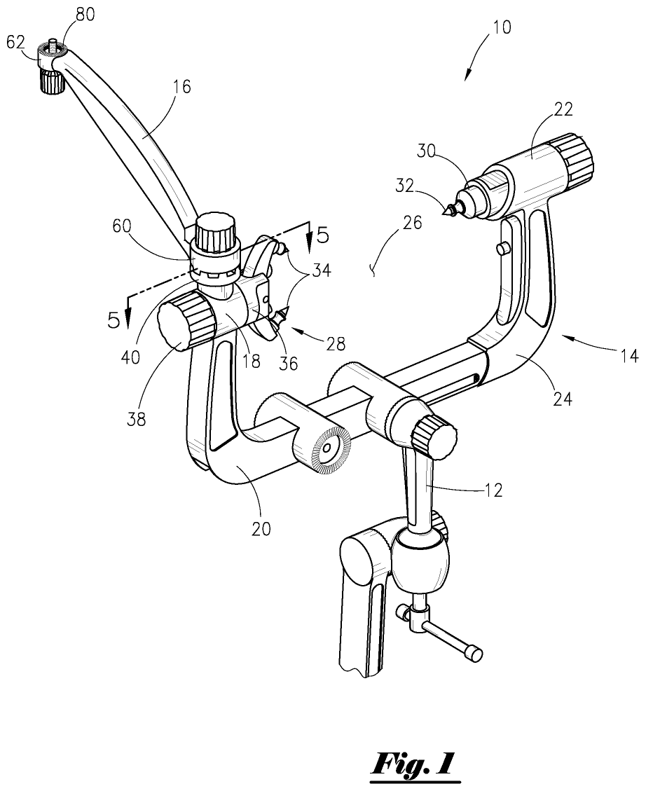

Disclosed herein is an apparatus, system, and method for providing a rigid connection between a localizing camera and a patient's head to facilitate image-guided surgery. A fixed camera head clamp includes a camera mount rigidly connected to a head clamp. The camera mount includes a camera receptacle configured to rigidly secure a localizing camera thereto such that the localizing camera is in a fixed position near the patient's head. The localizing camera may have a close view range, e.g., 1 cm to 75 cm, and a wide field of view, e.g., at least 120 degrees. The joints of the fixed camera head clamp provide rigid connections to reduce movement or backlash when the patient's head or body is moved or when the head clamp is adjusted. The fixed camera system eliminates the need to move the localizing camera around the operating room. The fixed camera system also eliminates the need for a dynamic reference frame. The fixed camera head clamp system also includes a drape configured to fit over the localizing camera such that the camera does not need to be removed and replaced after registering the patient with the localizing camera. Thus, the fixed camera system minimizes the error in conventional systems by reducing the relative motion between the camera and the patient, eliminating the dynamic reference frame, and improving the draping process. This results in improved navigation system accuracy. A camera mount system includes a camera mount secured to a patient's head and a localizing camera secured to a camera receptacle on the camera mount. The camera mount may be secured to the patient's head using a pin assembly. The camera mount system may be used to facilitate image-guided surgery in which the patient's head is mobile. FIGS. 1-8 and 10 illustrate exemplary embodiments of the fixed camera head clamp and system and the camera mount system, but numerous alternate embodiments of these devices and systems are described herein and will be readily apparent to skilled artisans after reviewing this disclosure.

With reference to FIG. 1, fixed camera head clamp 10 includes base support 12, frame 14, and camera mount 16. Base support 12 is configured for connection to one or more support members, which are directly or indirectly secured to a surgical table on which a patient is positioned. Frame 14 may have any shape, such as a C-shape, H-shape, U-shape, V-shape, W-shape, or any other shape providing a central space dimensioned to receive a patient's head. In one embodiment, frame 14 is U-shaped. Frame 14 may include first upper end 18 on fixed frame portion 20 and second upper end 22 on movable frame portion 24. Fixed frame portion 20 extends from base support 12 to first upper end 18. Fixed frame portion 20 is rigidly connected to base support 12. Movable frame portion 24 extends from fixed frame portion 20 to second upper end 22. A position of movable frame portion 24 is selectively adjustable relative to fixed frame portion 20 and base support 12. In one embodiment, movable frame portion 24 slidingly engages fixed frame portion 20. In a further embodiment, movable frame portion 24 may include a steel bearing to enable tight tolerances, eliminate wobble and backlash, and extend the life of the system. Frame 14 defines central space 26 between fixed frame portion 20 and movable frame portion 24.

First pin assembly 28 is secured to first upper end 18, and second pin assembly 30 is secured to second upper end 22 of frame 14. Second pin assembly 30 may include one or more pins extending into central space 26. For example, second pin assembly 30 may include one pin 32. First pin assembly 28 may include one or more pins extending into central space 26.

For example, first pin assembly 28 may include two pins 34. First pin assembly 28 and pins 34 are separated from second pin assembly 30 and pin 32 by central space 26.

First pin assembly 28 may include rotating base 36 and rotational lock 38. Pins 34 may be secured to rotating base 36 to selectively rotate pins 34. When in an engaged position, rotational lock 38 may prevent rotation of rotating base 36 thereby locking the position of each pin 34. In one embodiment, rotational lock 38 completely prevents rotation of rotating base 36 in the engaged position. In another embodiment, rotational lock 38 prevents rotation of pins 34 with zero backlash.

Referring to FIGS. 1-3, receptacle 40 is positioned at first upper end 18 of frame 14. In one embodiment, receptacle 40 is detachably secured to first upper end 18 with bolts 42, screws, or another connection mechanism. Bolts 42 may extend through openings 44 in receptacle 40 and engage a threaded inner surface in openings 46 in first upper end 18 to secure receptacle 40 to first upper end 18. Bottom surface 48 of receptacle 40 may have a shape that is reciprocal to the shape of the upper surface of first upper end 18. For example, first upper end 18 may have a rounded upper surface and bottom surface 48 of receptacle 40 may have a reciprocal concave shape. In an alternate embodiment, receptacle 40 may be integrally formed with first upper end 18 of frame 14.

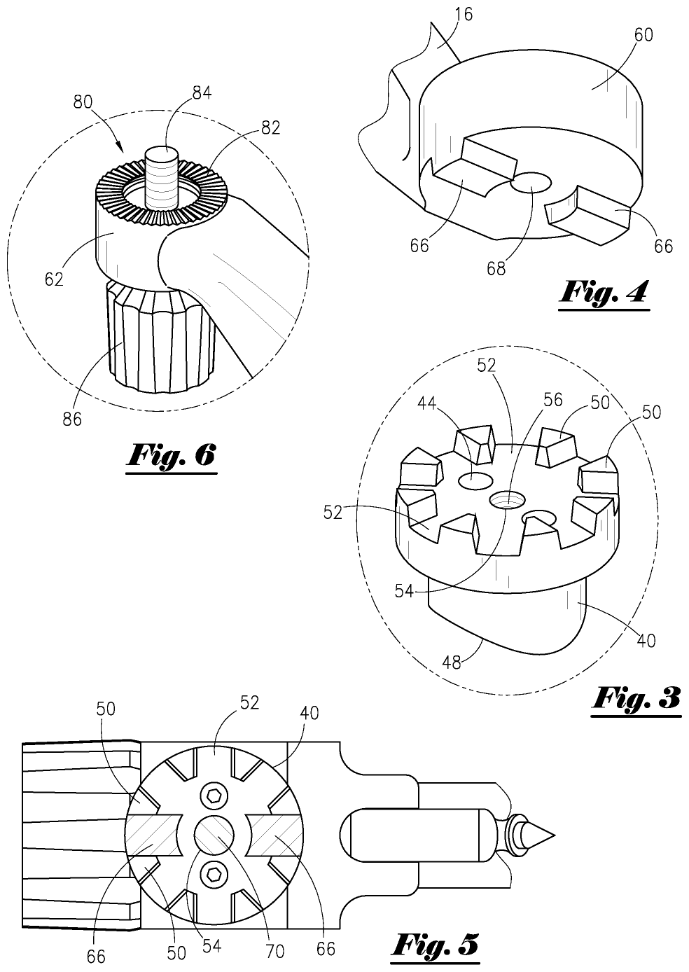

With reference now to FIG. 3, the upper surface of receptacle 40 may include a series of projections 50 separated by a series of receptacle spaces 52. In other words, receptacle spaces 52 are disposed between projections 50. In one embodiment, projections 50 form a dovetail design to ensure that camera mount 16 is rigidly connected to fixed frame portion 20. Receptacle 40 may further include bore 54 having threaded inner surface 56.

Referring now to FIGS. 1, 2, and 4, camera mount 16 extends from proximal end 60 to distal end 62. Proximal end 60 of camera mount 16 is selectively secured to receptacle 40. Proximal end 60 of camera mount 16 may include a receptacle lock configured to engage receptacle 40 to prevent rotation of camera mount 16 relative to frame 14. In one embodiment, the receptacle lock of proximal end 60 of camera mount 16 may include one or more lock projections 66 configured to engage receptacle spaces 52 on the upper surface of receptacle 40. The configuration of the series of projections 50 on receptacle 40 may allow for connection of camera mount 16 at a number of angles relative to frame 14 by positioning lock projections 66 in different receptacle spaces 52. Proximal end 60 may further include bore 68 configured to align with bore 54 of receptacle 40 when proximal end 60 engages receptacle 40 at all angles. The shape, length, and angle of the camera mount may vary in other embodiments.

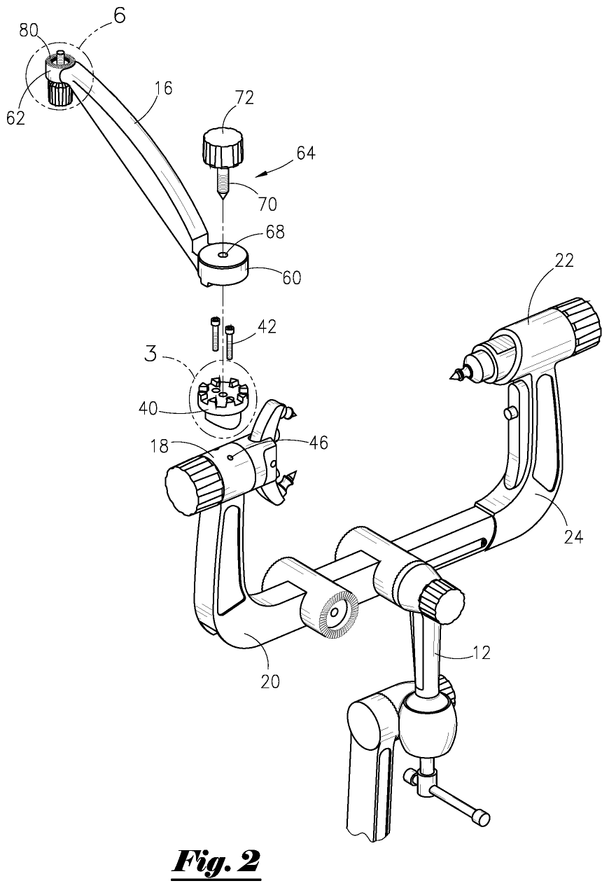

With reference to FIG. 2, fastener 64 may include bolt 70 and knob 72. Knob 72 is configured to rotate bolt 70. Bolt 70 of fastener 64 is configured to extend through bore 68 in proximal end 60 of camera mount 16 and to engage threaded inner surface 56 of bore 54 in receptacle 40. In this way, fastener 64 may secure proximal end 60 of camera mount 16 to receptacle 40 and prevent rotation of camera mount 16 relative to fixed frame portion 20.

FIG. 5 illustrates the connection of proximal end 60 of camera mount 16 to receptacle 40. Specifically, lock projections 66 of proximal end 60 are each disposed at least partially in one of the receptacle spaces 52 on the upper surface of receptacle 40. The interaction of lock projections 66 and projections 50 adjacent to the receptacle spaces 52 in which lock projections 66 are disposed prevent rotation of camera mount 16 relative to receptacle 40, and in turn, relative to frame 14. FIG. 5 also illustrates bolt 70 of fastener 64 disposed in bore 54 of receptacle 40 to secure proximal end 60 of camera mount 16 to receptacle 40.

With reference now to FIGS. 1, 2, and 6, distal end 62 of camera mount 16 includes camera receptacle 80. The upper surface of camera receptacle 80 may include a plurality of ridges 82 configured to lock a camera in place. In one embodiment, the plurality of ridges 82 may be formed of a starburst pattern, such as a deep-toothed starburst pattern. The plurality of ridges 82 may be disposed along an outer portion of the upper surface of camera receptacle 80. Camera receptacle 80 may also include threaded bolt 84 and knob 86. Knob 86 is configured to rotate threaded bolt 84. Threaded bolt 84 may extend upward from a central portion of the upper surface of camera receptacle 80. Threaded bolt 86 may extend upward beyond the plurality of ridges 82.

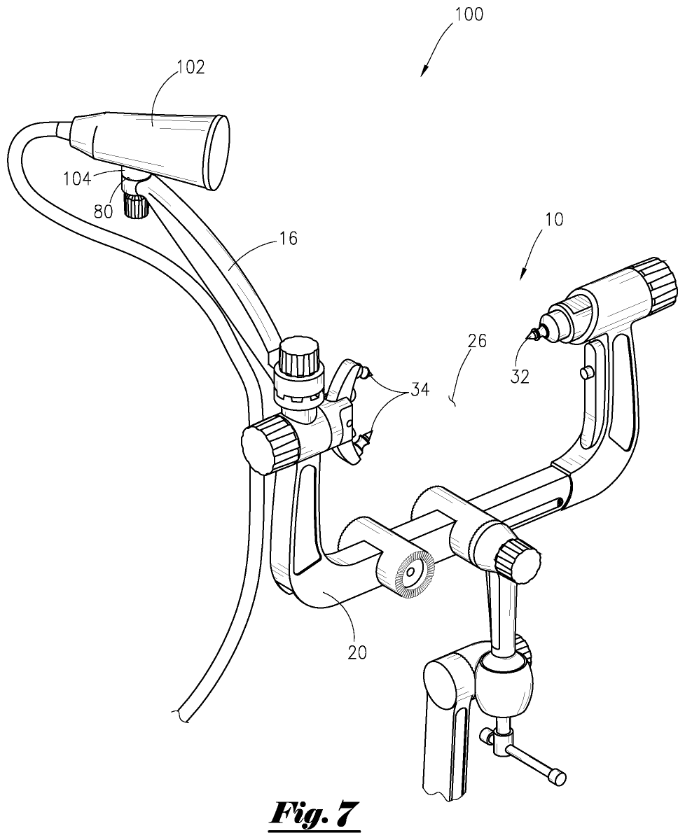

Referring now to FIG. 7, fixed camera head clamp system 100 includes head clamp 10 and camera 102. Camera 102 may include camera base 104. Camera base 104 may be secured to camera receptacle 80 of camera mount 16 to secure camera 102 to camera mount 16. In one embodiment, the plurality of ridges 82 of camera receptacle 80 has a shape that is reciprocal to the shape of a lower surface of camera base 104. Alternatively, camera base 104 and camera receptacle 80 may each have any other reciprocal shape. For example, a lower surface of camera base 104 may include one or more projections, and an upper surface of camera receptacle 80 may include one or more recesses configured to receive the projections of camera base 104. Numerous other reciprocal arrangements for camera base 104 and camera receptacle 80 will be readily apparent to skilled artisans. Using knob 86 (shown in FIG. 6), threaded bolt 84 may be secured within a bore of camera 102 to secure camera 102 to camera receptacle 80 with the plurality of ridges 82 preventing rotation of camera 102 relative to camera receptacle 80.

Camera 102 may be a localizing camera. In one embodiment, camera 102 may have an integrated accelerometer and gravitometer. Camera 102 may have a wide field of view. For example, camera 102 may have a field of view of at least approximately 120 degrees. Camera 102 may also have a close range. For example, camera 102 may have a range of approximately 1 cm-75 cm. In one embodiment, camera 102 may be a Mini Optical 3D, USB camera sold by Intellijoint Surgical.

In one embodiment, camera mount 16 may be formed of an I-shaped beam to eliminate or reduce deflection of camera mount 16 when camera 102 is attached to camera receptacle 80. Because camera 102 is rigidly secured to camera mount 16, which is in turn rigidly secured to fixed frame portion 20, there is reduced relative movement between camera 102 and a patient's head positioned in central space 26 between pin 32 and pins 34. In some embodiments, the rigid connections of fixed camera head clamp system 100 results in no relative movement between camera 102 and a patient's head positioned in central space 26.

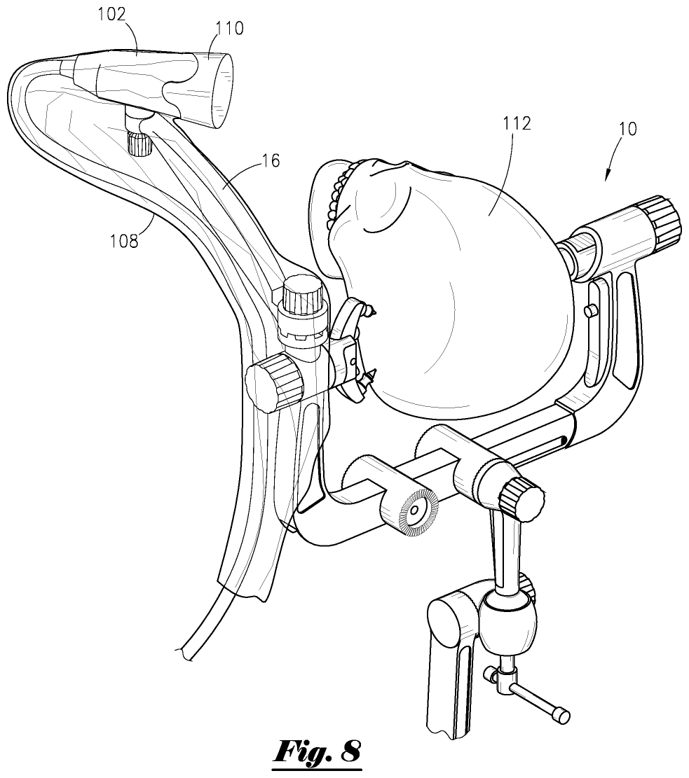

With reference to FIG. 8, fixed camera head clamp system 100 may further include drape 108 configured to cover camera 102 and camera mount 16 to provide a sterile field for surgery. System 100 may also include clip 110 configured to provide a smooth and secure fit of drape 108 over the lens of camera 102. After positioning drape 108 over camera 102, clip 110 may be secured over drape 108 at the forward end of camera 102 to hold drape 108 flat over the lens of camera 102. Clip 110 may be autoclavable. Camera 102 may be positioned 15 cm or less from the patient's head 112.

With reference now to FIGS. 1, 7, and 8, fixed camera head clamp system 100 may be used to immobilize a patient's head for image-guided brain surgery. In other embodiments, head clamp 10 and head clamp system 100 may be used to immobilize a patient's head for image-guided ENT surgery, dental surgery, plastic surgery, skull surgery, or any other surgery for which a patient's head is immobilized. Head clamp 10 may be attached to a surgical table in an operating room by securing base support 12 to the table. A user may slide movable frame portion 24 away from fixed frame portion 20 such that first upper end 18 and second upper end 22 move apart and central space 26 is enlarged. The patient may then be positioned on the surgical table with the patient's head 112 positioned in central space 26 of head clamp 10. A user may then slide movable frame portion 24 toward fixed frame portion 20 to move second upper end 22 closer to first upper end 18 until pins 34 and pin 32 engage the patient's head 112. First pin assembly 28 may be adjusted to set the desired rotational position of pins 34. Pins 34 and pin 32 may then be secured to the patient's head 112 to clamp the patient's head 112 in head clamp 10.

Camera mount 16 may then be connected to receptacle 40 at a desired angle relative to fixed frame portion 20. Lock projections 66 (shown in FIG. 4) of proximal end 60 of camera mount 16 may engage the receptacle spaces 52 (shown in FIG. 3) of receptacle 40 that coordinate with the desired angle. Bolt 70 may then be secured through bores 68 and 54 to secure camera mount 16 to receptacle 40 and fixed frame portion 20.

Camera 102 may then be attached to camera mount 16. Camera base 104 may be secured to camera receptacle 80 on camera mount 16 as described above. Camera 102 may be positioned such that a lens of camera 102 faces a direction that approximately parallel to camera mount 16. Drape 108 may then be positioned over camera 102 and camera mount 16. The patient's anatomy may then be registered by camera 102 using standard registration methods employed by surgical navigation systems. The patient may then be draped and the surgery may be performed.

During the surgery, camera 102 may transmit information to a surgical navigation system that generates and displays multiplanar, three-dimensional images of the patient's anatomy and the position of the surgeon's instruments on a monitor in the operating room. This system enables cranial surgical navigation without dynamic referencing because it provides a rigid connection between the patient's head 112 and camera 102. Error and inaccuracies are reduced or eliminated by system 100 because there is no need to move camera 102 around the operating room and camera 102 remains in a fixed position relative to the patient's head.

Because the sterile drape 108 is applied over the camera 102 while camera 102 remains rigidly fixed to the head clamp instead of removing and reattaching the dynamic referencing frame in conventional systems, fixed camera head clamp system 100 eliminates this additional step that introduces error or inaccuracies in conventional systems. The patient can be registered before or after the sterile drape is applied over camera 102.

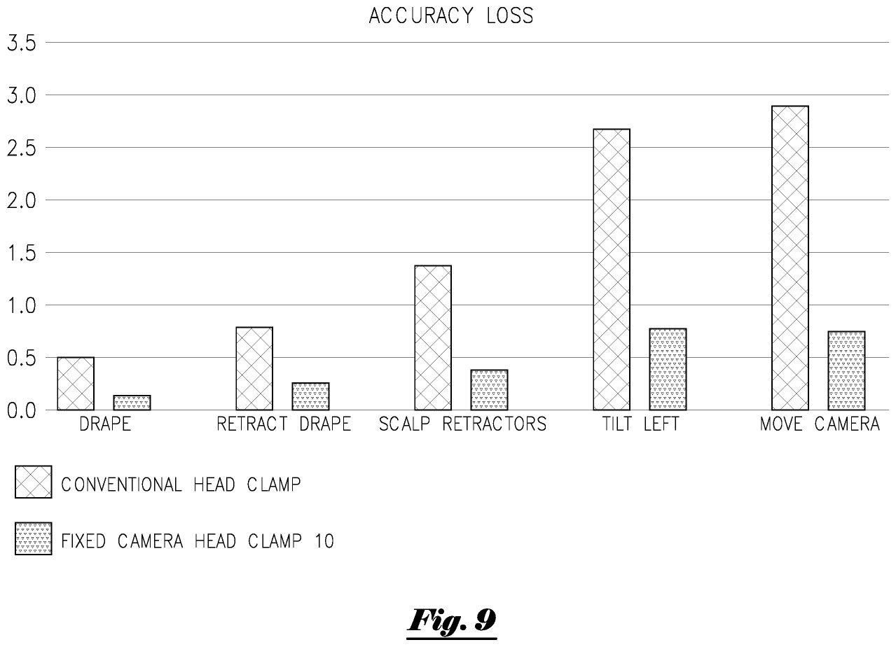

Referring now to FIG. 9, tests were conducted to determine the accuracy loss resulting from certain activities in the operating room using fixed camera head clamp 10 and system 100 in comparison to conventional head clamps. The process of applying sterilized drapes to the patient and the clamp system resulted in 0.5 mm of accuracy loss using a conventional head clamp, but only 0.1 mm of accuracy loss using fixed camera head clamp 10. Similarly, the process of retracting the drape resulted in 0.8 mm of accuracy loss using a conventional head clamp, but only 0.25 mm of accuracy loss using fixed camera head clamp 10. Use of scalp retractors resulted in 1.4 mm of accuracy loss using a conventional head clamp, but only 0.35 mm of accuracy loss using fixed camera head clamp 10. Tilting the patient's head resulted in 2.6 mm of accuracy loss using a conventional head clamp, but only 0.75 mm of accuracy loss using fixed camera head clamp 10. Moving the camera resulted in 2.9 mm of accuracy loss using a conventional head clamp, but only 0.75 mm of accuracy loss using fixed camera head clamp 10. These tests demonstrated the improved accuracy of head clamp 10 and system 100 over conventional head clamps.

In an alternate embodiment, camera mount 16 may be attached to any existing head clamp. In this embodiment, proximal end 60 of camera mount 16 may be configured to be secured to a fitting on the existing head clamp. For example, the fitting of the existing head clamp may include the same projections and recesses as the upper surface of receptacle 40 described herein. In another example, proximal end 60 may include a plurality of ridges, such as a starburst configuration, that is configured to be secured to a receptacle of the existing head clamp. In other embodiments, a small localizing camera may be directly attached to any immobilizing device, such as a head frame (including existing head frames), using any affixation mechanism, including but not limited to a clamp, a vice grip, adhesive, tape. In yet another embodiment, a localizing camera may be fixed directly to the patient's head.

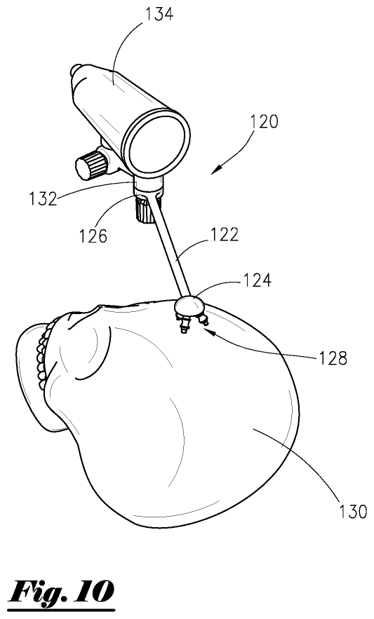

With reference now to FIG. 10, camera mount system 120 may include camera mount 122 extending from proximal end 124 to distal end 126. Proximal end 124 of camera mount 122 may be attached to pin assembly 128, which is directly attached to a patient's head 130. Pin assembly 128 includes two or more pins to prevent rotation of camera mount 122 relative to the patient's head 130. Alternatively, proximal end 124 of camera mount 122 may be secured to the patient's head 130 with any securing mechanism that is configured to support the weight and prevent rotation of camera mount 122. Camera receptacle 132 may be attached to distal end 126 of camera mount 122. Localizing camera 134 may be secured to camera receptacle 132. In an alternate embodiment, the camera receptacle may be integrally formed with distal end 126 of camera mount 122, and may include a plurality of ridges, similar to plurality of ridges 82 of camera receptacle 80 shown in FIG. 6. Camera mount system 120 reduces the relative motion between camera 134 and the patient's head 130 for image-guided surgery in which the patient's head is not immobilized, i.e., no head clamp is used. Camera mount system 120 provides a rigid connection between camera 134 and the patient's head 130. Camera mount system 120 may be used by attaching proximal end 124 of camera mount 122 to pin assembly 128, then attaching pin assembly 128 to the patient's head 130. Alternatively, pin assembly 128 may first be attached to the patient's head 130, and proximal end 124 of camera mount 122 may then be attached to pin assembly 128. Finally, localizing camera 134 may be secured to camera receptacle 132.

Except as otherwise described or illustrated, each of the components in this device may be formed of aluminum, steel, another metal, plastic, or any other durable material. Each device described in this disclosure may include any combination of the described components, features, and/or functions of each of the individual device embodiments. Each method described in this disclosure may include any combination of the described steps in any order, including the absence of certain described steps and combinations of steps used in separate embodiments. Any range of numeric values disclosed herein includes any subrange therein. Plurality means two or more.

While preferred embodiments have been described, it is to be understood that the embodiments are illustrative only and that the scope of the invention is to be defined solely by the appended claims when accorded a full range of equivalents, many variations and modifications naturally occurring to those skilled in the art from a review hereof.

We claim:

1. A head clamp for facilitating image-guided surgery, comprising a camera mount for rigidly securing a localizing camera to the head clamp.

2. The head clamp of claim 1, further comprising:
a base support;
a frame secured to an upper end of the base support, wherein the frame includes a first upper end and a second upper end, with a receptacle disposed at the first upper end; wherein the frame defines a central space dimensioned to receive a patient's head;
a first pin assembly secured to the first upper end of the frame; and
a second pin assembly secured to the second upper end of the frame;
wherein the camera mount extends from a proximal end to a distal end, wherein the proximal end of the camera mount is selectively secured to the receptacle of the frame, wherein the distal end of the camera mount includes a camera receptacle configured to receive a localizing camera.

3. The head clamp of claim 2, wherein the frame includes a fixed frame portion and a movable frame portion, wherein the movable frame portion is selectively movable relative to the fixed frame portion and the base support.

4. The head clamp of claim 3, wherein the fixed frame portion extends from the base support to the first upper end; and wherein the movable frame portion extends from the fixed frame portion to the second upper end.

5. The head clamp of claim 4, wherein the first pin assembly includes two pins and the second pin assembly includes one pin.

6. The head clamp of claim 5, wherein the first pin assembly further includes a rotating base and a rotational lock; wherein the two pins of the first pin assembly are secured to the rotating base to selectively rotate the two pin; wherein in an engaged position the rotational lock prevents rotation of the rotating base.

7. The head clamp of claim 4, wherein the proximal end of the camera mount includes a receptacle lock configured to engage the receptacle of the frame to prevent rotation of the camera mount relative to the fixed frame portion of the frame.

8. The head clamp of claim 7, wherein the receptacle of the frame includes a series of projections and a series of receptacle spaces disposed between the projections.

9. The head clamp of claim 8, wherein the receptacle lock of the proximal end of the camera mount includes one or more lock projections dimensioned to fit into one or more of the receptacle spaces.

10. The head clamp of claim 9, further comprising a fastener including a bolt and a knob configured to secure the proximal end of the camera mount to the receptacle of the frame.

11. The head clamp of claim 10, wherein the bolt of the fastener is configured to extend through a bore in the proximal end of the camera mount and to engage a bore of the receptacle of the frame.

12. The head clamp of claim 4, wherein the camera receptacle at the distal end of the camera mount includes an upper surface having a plurality of ridges.

13. The head clamp of claim 12, wherein the camera receptacle further includes a threaded bolt and a knob, wherein the threaded bolt extends upward beyond the plurality of ridges.

14. The head clamp of claim 4, wherein the receptacle of the frame is detachably secured to the first upper end of the frame.

15. A fixed camera head clamp system for facilitating image-guided surgery, comprising:
a base support;
a frame secured to an upper end of the base support, wherein the frame includes a first upper end and a second upper end, with a receptacle disposed at the first upper end; wherein the frame defines a central space dimensioned to receive a patient's head;
a first pin assembly secured to the first upper end of the frame;
a second pin assembly secured to the second upper end of the frame;
a camera mount extending from a proximal end to a distal end, wherein the proximal end of the camera mount is selectively secured to the receptacle of the frame, wherein the distal end of the camera mount includes a camera receptacle; and
a localizing camera selectively attached to the camera receptacle.

16. The fixed camera head clamp system of claim 15, further comprising a drape configured to cover the localizing camera while the localizing camera is attached to the camera receptacle.

17. The fixed camera head clamp system of claim 15, wherein the localizing camera has a field of view of at least approximately 120 degrees.

18. The fixed camera head clamp system of claim 15, wherein the localizing camera has a view range of approximately 1 cm to 75 cm.

19. The fixed camera head clamp system of claim 15, wherein the localizing camera is positioned approximately 15 cm or less from the first pin assembly when attached to the camera receptacle.

20. A method of immobilizing a patient's head to facilitate image-guided surgery, comprising the steps of:
a) placing a patient's head in an immobilizing device; and
b) attaching a localizing camera to the immobilizing device.

21. The method of claim 20, wherein the immobilizing device comprises a head clamp including a camera mount, and wherein step (b) further includes attaching the localizing camera to the camera mount to rigidly secure the localizing camera to the head clamp.

22. The method of claim 21, wherein the head clamp further comprises: a base support; a frame secured to an upper end of the base support, wherein the frame includes a first upper end and a second upper end, with a receptacle disposed at the first upper end; wherein the frame defines a central space dimensioned to receive a patient's head; a first pin assembly secured to the first upper end of the frame; and a second pin assembly secured to the second upper end of the frame; wherein the camera mount extends from a proximal end to a distal end, wherein the proximal end of the camera mount is selectively secured to the receptacle of the frame, wherein the distal end of the camera mount includes a camera receptacle.

23. The method of claim 22, wherein step (a) further comprises
ii) attaching the base support and the frame to the surgical table;
iii) positioning a patient on the surgical table with the patient's head positioned in the central space of the frame;
iv) clamping the patient's head in the head clamp using the first pin assembly and the second pin assembly;
v) attaching the camera mount to the frame by securing the proximal end of the camera mount to the receptacle of the frame;

24. The method of claim 23, wherein step (b) further comprises:
vi) attaching the localizing camera to the camera receptacle at the distal end of the camera mount; and
vii) positioning a drape over the localizing camera and the camera mount.

25. The method of claim 24, wherein the frame includes a fixed frame portion and a movable frame portion, wherein the movable frame portion is selectively movable relative to the fixed frame portion and the base support, wherein the method further includes the step of:
sliding the movable frame portion away from the fixed frame portion to expand the central space before the patient's head is positioned in the central space;
sliding the movable frame portion toward the fixed frame portion to adjust the size of the central space when the patient's head is positioned in the central space.

26. The method of claim 24, wherein the proximal end of the camera mount includes a receptacle lock, and wherein step (e) further includes engaging the receptacle of the frame with the receptacle lock of the proximal end of the camera mount to prevent rotation of the camera mount relative to the frame.

27. A camera mount system for facilitating image-guided surgery, comprising:
a camera mount extending from a proximal end to a distal end, wherein the proximal end of the camera mount is configured for selective attachment to a patient's head, wherein the distal end of the camera mount includes a camera receptacle; and
a localizing camera selectively attached to the camera receptacle.

28. The camera mount system of claim 27, wherein the proximal end of the camera mount is selectively attached to the patient's head through a pin assembly.

29. The camera mount system of claim 28, wherein the pin assembly includes two or more pins for direct attachment to the patient's head.

30. The camera mount system of claim 27, wherein the camera receptacle of the camera mount includes an upper surface having a plurality of ridges.

31. A method of using a localizing camera to facilitate image-guided surgery, comprising:
securing a localizing camera in a fixed position relative to a patient's head.

32. The method of claim 31, wherein the method further includes:
a) attaching a proximal end of a camera mount to the patient's head; and
b) attaching the localizing camera to a distal end of the camera mount.

33. The method of claim 32, wherein step (b) further includes attaching the localizing camera to a camera receptacle at the distal end of the camera mount, wherein the camera receptacle is configured to attach the localizing camera to the camera mount in two or more directions.

34. The method of claim 33, wherein the camera receptacle includes an upper surface having a plurality of ridges.
